(19) United States
(12) Patent Application Publication
(10) Pub. No.: US 2023/0410307 A1
(43) Pub. Date: Dec. 21, 2023

(54) METHOD AND SYSTEM FOR VISUALIZATION

(71) Applicant: KONINKLIJKE PHILIPS N.V., EINDHOVEN (NL)

(72) Inventors: HANNES NICKISCH, HAMBURG (DE); HOLGER SCHMITT, LUETJENSEE (DE); CLAAS BONTUS, HAMBURG (DE); RAFAEL WIEMKER, KISDORF (DE)

(21) Appl. No.: 18/037,586

(22) PCT Filed: Nov. 22, 2021
(86) PCT No.: PCT/EP2021/082456

§ 371 Date: May 18, 2023

Related U.S. Application Data
(60) Provisional application No. 63/119,308, filed on Nov. 30, 2020.

(51) Int. Cl. G06T 7/00; A61B 6/00; G06T 7/11; G06T 7/60; G16H 30/40

(52) U.S. Cl. CPC G06T 7/0014; A61B 6/507; A61B 6/503; G06T 7/11; G06T 7/60; G16H 30/40; G06T 2207/20021; G06T 2207/30048; G06T 2207/30104; G06T 2207/10081

(57) ABSTRACT

A method for visualization may include: obtaining data of a first perfusion measure of myocardial tissues of a patient; obtaining data of a geometry of a coronary artery of the patient; obtaining data of a second perfusion measure of the coronary artery; obtaining data of a flow impediment measure along the coronary artery based on the data of the second perfusion measure of the coronary artery; and visualizing, on a single image, the first perfusion measure of the myocardial tissues and the coronary artery, the coronary artery being overlaid with the first perfusion measure on the single image, the visualized coronary artery representing the geometry of the coronary artery and the flow impediment measure along the coronary artery.

The present disclosure generally relates to methods and systems for visualization, in particular, for visualization of myocardial perfusion measure and coronary artery.

### BACKGROUND

Conventionally, Computed Tomography (CT) and, in particular, Coronary Computed Tomography Angiography (CCTA) have been used to assess coronary arteries in terms of their morphology and geometry and predict the associated future risk for Major Adverse Cardiovascular Events (MACE). Spectral CT acquisitions have potential to allow to determine local myocardial perfusion by directly measuring an iodine content in the myocardial tissue, which yields a functional assessment of heart muscle. There is a need for a method to allow for a comprehensive risk assessment.

The description provided in the background section should not be assumed to be prior art merely because it is mentioned in or associated with the background section. The background section may include information that describes one or more aspects of the subject technology.

### SUMMARY

One embodiment of the present disclosure may provide a method for visualization. The method may include: obtaining data of a first perfusion measure of myocardial tissues of a patient; obtaining data of a geometry of a coronary artery of the patient; obtaining data of a second perfusion measure of the coronary artery; obtaining data of a flow impediment measure along the coronary artery based on the data of the second perfusion measure of the coronary artery; and visualizing, on a single image, the first perfusion measure of the myocardial tissues and the coronary artery, the coronary artery being overlaid with the first perfusion measure on the single image, the visualized coronary artery representing the geometry of the coronary artery and the flow impediment measure along the coronary artery.

Another embodiment of the present disclosure may provide a visualization system. The visualization system may include: a memory that stores a plurality of instructions; and processor circuitry that couples to the memory. The processor circuitry may be configured to execute the instructions to: obtain data of a first perfusion measure of myocardial tissues of a patient; obtain data of a geometry of a coronary artery of the patient; obtain data of a second perfusion measure of the coronary artery; obtain data of a flow impediment measure along the coronary artery based on the data of the second perfusion measure of the coronary artery; and visualize, on a single image, the first perfusion measure of the myocardial tissues and the coronary artery, the coronary artery being overlaid with the first perfusion measure on the single image, the visualized coronary artery representing the geometry of the coronary artery and the flow impediment measure along the coronary artery.

Another embodiment of the present disclosure may provide a non-transitory computer-readable medium having one or more executable instructions stored thereon, which, when executed by processor circuitry, cause the processor circuitry to perform a method for visualization. The method may include: obtaining data of a first perfusion measure of myocardial tissues of a patient; obtaining data of a geometry of a coronary artery of the patient; obtaining data of a second perfusion measure of the coronary artery; obtaining data of a flow impediment measure along the coronary artery based on the data of the second perfusion measure of the coronary artery; and visualizing, on a single image, the first perfusion measure of the myocardial tissues and the coronary artery, the coronary artery being overlaid with the first perfusion measure on the single image, the visualized coronary artery representing the geometry of the coronary artery and the flow impediment measure along the coronary artery.

### DRAWINGS

FIG. 1 illustrates an example of a three dimensional surface rendering of four chambers of a human heart according to one embodiment of the present disclosure.

FIG. 2A illustrates a schematic diagram of a system according to one embodiment of the present disclosure.

FIG. 2B illustrates a schematic diagram of a system according to one embodiment of the present disclosure.

FIG. 3A illustrates a linear vessel tapering model used to predict a deviation of a vessel effective radius from a linearly decaying robust fit to an actual vessel radius according to one embodiment of the present disclosure.

FIG. 3B illustrates percent stenosis along a centerline of coronary arteries according to one embodiment of the present disclosure.

FIG. 4A illustrates a left ventricular bull's eye plot sector according to one embodiment of the present disclosure.

FIG. 4B illustrates myocardial slices and sectors corresponding to FIG. 4A.

FIG. 5 schematically illustrates aspects of constructing a target shape, in the form of a volumetric bull's eye plot (VBEP) according to one embodiment of the present disclosure.

FIG. 6 illustrates a visualized perfusion measure and visualized coronary arteries using a bull's eye plot according to one embodiment of the present disclosure.

FIG. 7 illustrates a flowchart of a method according to one embodiment of the present disclosure.

### EMBODIMENTS

The description of illustrative embodiments according to principles of the present disclosure is intended to be read in connection with the accompanying drawings, which are to be considered part of the entire written description. In the description of embodiments of the disclosure disclosed herein, any reference to direction or orientation is merely intended for convenience of description and is not intended in any way to limit the scope of the present disclosure. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the disclosure are illustrated by reference to the exemplified embodiments. Accordingly, the disclosure expressly should not be limited to such exemplary embodiments illustrating some possible non-limiting combination of features that may exist alone or in other combinations of features; the scope of the disclosure being defined by the claims appended hereto.

This disclosure describes the best mode or modes of practicing the disclosure as presently contemplated. This description is not intended to be understood in a limiting sense, but provides an example of the disclosure presented solely for illustrative purposes by reference to the accompanying drawings to advise one of ordinary skill in the art of the advantages and construction of the disclosure. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

The present disclosure may visualize, on a single image, a perfusion measure of myocardial tissues and a coronary artery, where the coronary artery is overlaid with the perfusion measure on the single image, and the visualized coronary artery may represent a geometry of the coronary artery and a flow impediment measure along the coronary artery. The perfusion measure of the myocardial tissues allows for quantifying local blood and oxygen supply, as well as perfusion defects of the myocardium. The geometry of the coronary artery may relate the observed perfusion and the blood supply as well, and may be used to compute a common coordinate system. The flow impediment measure along the coronary artery may then be used to quantify a local effect of the coronary geometry on an actual blood flow. In one example, these three items (i.e., the perfusion measure of myocardial tissues, the geometry of the coronary artery, and the flow impediment measure along the coronary artery) may be computed from a single spectral CCTA scan and mapped into a variation on a common bull's eye visualization, allowing a user to judge the relative contribution of each of the complementary aspects in a unified graphical figure.

In some embodiments of the present disclosure, because the three items (i.e., the perfusion measure of myocardial tissues, the geometry of the coronary artery, and the flow impediment measure along the coronary artery) may be aggregated into a common representation suitable for visualization and predictive analytics, a comprehensive risk assessment may be achieved more efficiently. In addition, in some embodiments of the present disclosure, the representation may be easily mappable to a plane for quick visual inspection, reporting and documentation purposes. In some embodiments of the present disclosure, the representation may be synchronized with standard reporting schemes and population models in order to allow for transferrable statements.

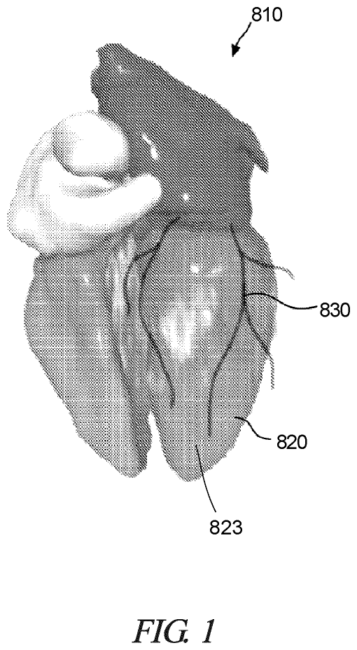

FIG. 1 illustrates a three dimensional (3D) surface rendering of four chambers of a human heart 810, including a myocardium wall 820 (e.g., a left ventricle in the illustrated example) and a portion of coronary arteries 830 (i.e., blood vessels surrounding the heart 810). The muscle tissues that form the myocardium wall 820 are called myocardium, or myocardial tissues 823. As well as any other muscle tissue, the heart muscle tissues needs oxygen to operate. Oxygen may be supplied by the coronary arteries 830.

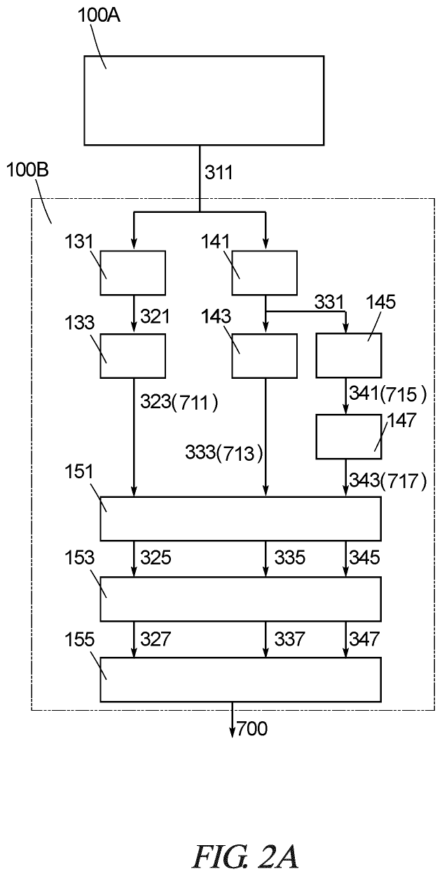

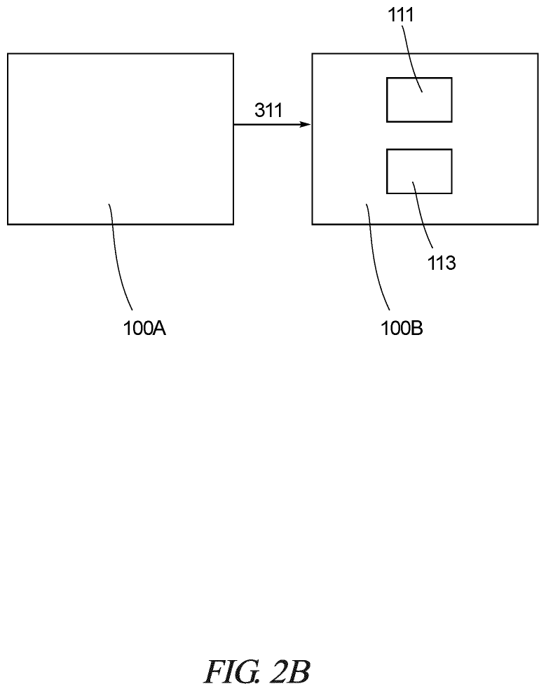

FIG. 2A illustrates a schematic diagram of a system according to one embodiment of the present disclosure. As shown in FIG. 2A, the system may include an imaging device 100A and a processing device 100B.

The imaging device 100A may obtain image data 311 of a patient. In some embodiments, the imaging device 100A may include a CT imaging device, and the image data 311 may include CT angiography data of the patient. The CT imaging device may obtain spectral computed tomography volumetric image data organized in voxels, e.g., spectral CCTA data, by way of a spectral acquisition and a tomographic reconstruction.

The volumetric image data may include a contrast-enhanced volumetric image of a cardiac region in the patient's body and a baseline volumetric image of the cardiac region, where the contrast-enhanced volumetric image may convey anatomical information regarding coronary artery anatomy of the patient. In the method, an iodine contrast agent may be used.

As shown in FIG. 2B the processing device 100B may include processor circuitry 111 and a memory 113. The memory 113 may store a plurality of instructions. The processor circuitry 111 may couple to the memory 113 and may be configured to execute the instructions. The processing device 100B may receive the image data 311 of the patient from the imaging device 100A.

As shown in FIG. 2A, the processing device 100B may include a plurality of function blocks 131, 133, 141, 143, 145, 147, 151, 153, and 155.

With reference to FIG. 2A, the block 131 may segment the image data 311 of the patient in accordance with a segment model of the myocardium wall 820 to provide segmented data 321 of the myocardium wall 820. The segmentation may be conducted manually by the user or by (semi)automatic segmentation. In one example of an automatic segmentation of the myocardium wall 820, the myocardium wall 820 may be automatically segmented using a convolutional neural network (CNN) trained on manually annotated data. A method for segmentation is described, for example, in Ecabert O, at al., Automatic model-based segmentation of the heart in CT images, IEEE Trans Med Imaging., 1189, (2008), which is incorporated herein by reference.

The block 133 may obtain data 323 of a first perfusion measure 711 of the myocardial tissues 823 of the patient. The obtaining of the data 323 of the first perfusion measure 711 of the myocardial tissues 823 may be based on the segmented data 321 of the myocardium wall 820. The data 323 of the first perfusion measure 711 may be contained, for example, in a three dimensional volumetric image.

In the example shown in FIG. 2A, the data 323 of the first perfusion measure 711 may be obtained by using a static spectral CT perfusion image. However, data of a first perfusion measure of myocardial tissues may be obtained by using other kind of images as well, for example, using positron emission tomography (PET), magnetic resonance (MR) perfusion images, or dynamic rest/stress CT perfusion images with a perfusion measure e.g. rest/stress difference.

With reference to FIG. 2A, the block 141 may segment the image data 311 of the patient in accordance with a segment model of the coronary arteries 830 to provide segmented data 331 of the coronary arteries 830. For example, the block 141 may be adapted to automatically extract vessel centerlines of the coronary arteries 830 and lumina within an image. Various algorithms for automatic or semiautomatic tracing of centerlines may be used. Any method for tracking the centerlines of the coronary vessels can be used. A method for extracting centerlines for coronary arteries is described, for example, in D. Lesage, et al., A Review of 3D Vessel Lumen Segmentation Techniques Models, Features and Extractions Schemes, Medical Image Analysis, 819, (2009), which is incorporated herein by reference. Another method for extracting centerlines for coronary arteries is described, for example, in Wolterink J M, et al., Coronary artery centerline extraction in cardiac CT angiography using a CNN-based orientation classifier, Med Image Anal. 46 (2019), which is incorporated herein by reference. A method for segmentation for coronary arteries is described, for example, in Billow T., et al. A general framework for tree segmentation and reconstruction from medical volume data, Med. Image Comput. Comput. Assist. Intervent. 533, (2004), which is incorporated herein by reference.

The block 143 may obtain data 333 of a geometry 713 of the coronary arteries 830 based on the segmented data 331 of the coronary arteries 830. For example, the geometry 333 of the coronary arteries 830 may be represented in terms of the centerlines of the coronary arteries 830 and the lumina of the coronary arteries 830.

An image used to obtain the data 333 of the geometry 713 of the coronary arteries 830 may be the same as, or may be different from, an image used to obtain the data 323 of the first perfusion measure 711 of the myocardial tissues 823. Instead of using the CCTA scan, the data 333 of the geometry 713 of the coronary arteries 830 may be obtained by using a different angiographic modality to extract coronary arteries or complement and refine the coronary arteries.

With reference to FIG. 2A, the block 145 may obtain data 341 of a second perfusion measure 715 of the coronary arteries 830. The obtaining of the data 341 of the second perfusion measure 715 of the coronary arteries 830 may be based on the segmented data 331 of the coronary arteries 830. The second perfusion measure 715 of the coronary arteries 830 may correspond to a blood flow of the coronary arteries 830. A blood flow of the coronary arteries 830 may be a functional quantity providing information about the perfusion downstream.

The block 147 may obtain data 343 of the flow impediment measure 717 along the coronary arteries 830 based on the data 341 of the second perfusion measure 715 of the coronary arteries 830. In some embodiments, the obtaining of the data 323 of the first perfusion measure 711, the obtaining of the data 333 of the geometry 713 of the coronary arteries 830, and the obtaining of the data 343 of the flow impediment measure 717 along the coronary arteries 830 may be based on the same coronary computed tomography angiography data.

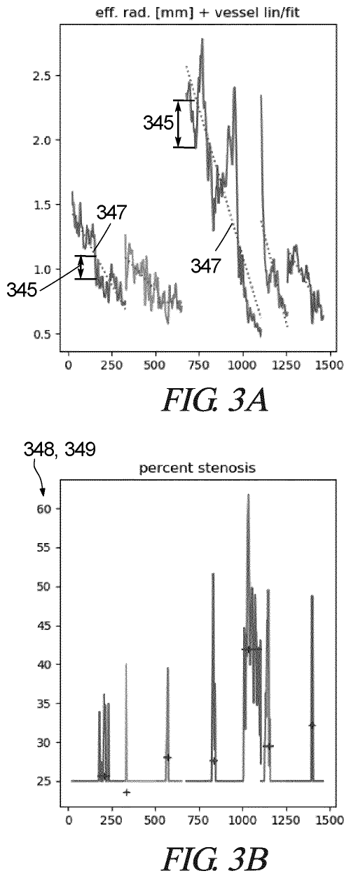

With reference to FIGS. 2A and 3A, in some embodiments, for obtaining the data 343 of the flow impediment measure 717, the block 147 may determine a flow deviation 345 of the second perfusion measure 715 from a reference value 347 along the coronary arteries 830. Values represented by solid lines in FIG. 3A may correspond to the second perfusion measure 715 of the coronary arteries 830. In one example shown in FIG. 3A, the flow deviation 345 may be determined by robustly fitting a linear tapering model in the effective radius domain along the centerline. The linear model cross-sectional area serves as the reference value 347. Then, the block 147 may process a stenosis assessment 348 (see FIG. 3B) along the coronary arteries 830 based on the determined flow deviation 345 to obtain the flow impediment measure 717. In one example, as shown in FIG. 3B, processing the stenosis assessment 348 along the coronary arteries 830 may include determining a plurality of stenosis rates 349 (e.g., percent stenosis, which may be the relative local loss in cross-sectional area) along the coronary arteries 830 based on the flow deviation 345. The stenosis rates 349 may include a measure to quantify the local flow impediment induced by a coronary stenosis. In FIG. 3B, peaks may indicate strong narrowings of the coronary arteries 830. Then, the block 147 may process a cumulative sum of the stenosis rates 349 to obtain the data 343 of the flow impediment measure 717 of the coronary arteries 830.

In some embodiments, instead of processing the cumulative loss in a cross-sectional area, data of a flow impediment measure may be obtained by using other measures such as cumulative loss in radius/diameter, pressure drop (fractional flow reserve (FFR)), volumetric blood flow, blood flow velocity, etc. Instead of the linear tapering model used to compute the cumulative loss in a cross-sectional area, a more advanced vessel tapering model can be used. Additional information, such as population statistics, prior images of the same patient, etc. may be used to obtain data of a flow impediment measure.

With reference to FIG. 2A, the block 151 may reformat the data 323 of the first perfusion measure 711 of the myocardial tissues 823, the data 333 of the geometry 713 of the coronary arteries 830, and the data 343 the of the flow impediment measure 717 along the coronary arteries 830. In some embodiments, the reformatting may be made for visualization on a volumetric bull's eye plot (VBEP).

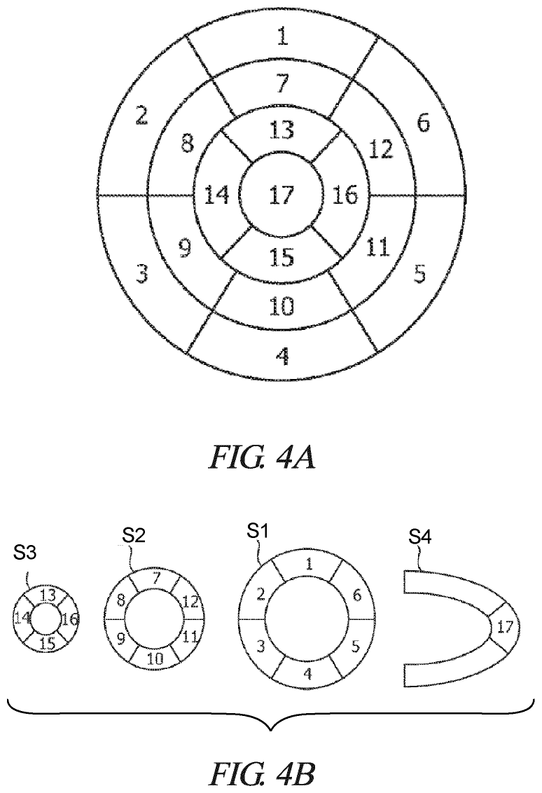

FIG. 4A schematically illustrates a left ventricular bull's eye plot sectors, and FIG. 4B illustrates the corresponding myocardial slices and sectors. A standardized myocardial segmentation for tomographic imaging of the heart has been proposed by the American Heart Association (AHA). The AHA proposal is described, for example, in M. Cerqueira, et al., Standardized Myocardial Segmentation and Nomenclature for Tomographic Imaging of the Heart, 539, (2002), which is incorporated herein by reference. As shown in FIGS. 4A and 4B, AHA recommends dividing the left ventricle into equal thirds perpendicular to the long axis of the heart to generate three slices of the left ventricle: the circular basal slice S1 comprising sectors 1 to 6, the mid-cavity slice S2 comprising sectors 7 to 12, and the apical short axis slice S3 comprising sectors 13 to 16. The last sector, the apex 17, is shown in a vertical long axis slice S4.

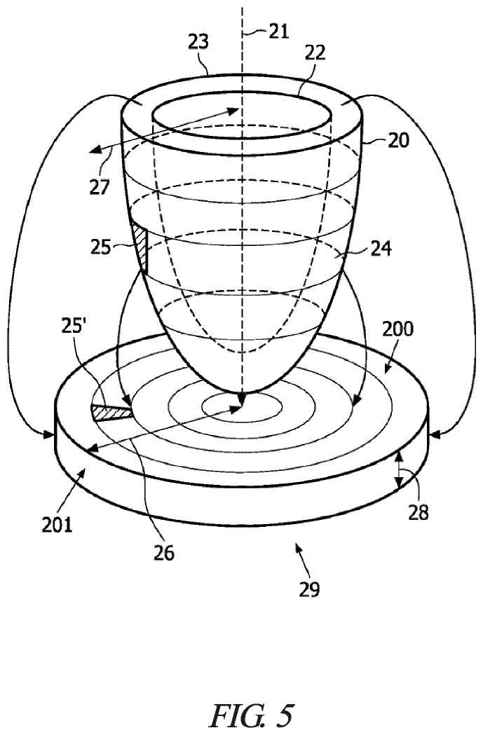

FIG. 5 schematically illustrates aspects of constructing a target shape 201, in the form of a volumetric bull's eye plot. The left ventricle including the slices S1-S4 of FIG. 4B may correspond to a reference shape 20 of FIG. 5, and the bull's eye plot shown in FIG. 4A may be visualized based on the target shape 201 of FIG. 5.

With reference to FIGS. 2A and 5, the block 151 may reformat the data 323 of the first perfusion measure 711 of the myocardial tissues 823, the data 333 of the geometry 713 of the coronary arteries 830, and the data 343 the of the flow impediment measure 717 along the coronary arteries 830, to fit the reference shape 20 (see FIG. 5). Then, the block 151 may obtain reformatted data 325 of the myocardial tissues 823, reformatted data 335 of the geometry 713 of the coronary arteries 830, and reformatted data 345 of the flow impediment measure 717 along the coronary arteries 830.

As shown in FIG. 5, the reference shape 20 is in the form of a truncated ellipsoid. In the reformatting of the data 323, 333, and 343, which may be voxel data, the true anatomical location of the data may be fitted to match the reference shape 20. The segmentation may result in the positioning of the inner and outer reference surfaces 22 and 23.

The reference shape 20 includes the long axis 21 of the left ventricle, the inner reference surface 22 (e.g., the inner wall or endocardium) and the outer reference surface 23 (e.g., the outer wall or epicardium).

With reference to FIGS. 2A and 5, the block 153 may then map, to the target shape 201, the reformatted data 325 of the myocardial tissues 823, the reformatted data 335 of the geometry 713 of the coronary arteries 830, and the reformatted data 345 of the flow impediment measure 717 along the coronary arteries 830. Then, the block 153 may obtain mapped data 327 of the myocardial tissues 823, mapped data 337 of the geometry 713 of the coronary arteries 830, and mapped data 347 of the flow impediment measure 717 along the coronary arteries 830. The target shape 201 shown in FIG. 5 may be defined by at least a first target surface 29 and a second target surface 200. The mapping of the data 325, 335, and 345 may include mapping data from the inner reference surface 22 to the first target surface 29 and from the outer reference surface 23 to the second target surface 200. In some embodiments, the data 325, 335, and 345 may be mapped from the set of voxel values to the target shape 201 in the form of a cylinder. In connection with FIG. 5, each contour 24 of the reference shape 20 may constitute a section of the reference shape 20, and the contours 24 are projected to a single concentric ring of a two dimensional (2D) plane 26, in the form of a 2D plane of a cross-section of the target shape 201. In FIG. 5, the mapping is illustrated by mapping of the section 25 on the reference shape 20 to the section 25′ on the 2D plane of the target shape 201. The mapping may be continuous, so no quantification of the data in the section 25 on the reference shape 20 may be made in connection with the mapping to the section 25′ on the target shape. The sections are shown purely for illustrative reasons, not in order to indicate a quantification of the data.

The direction extending along the inter-surface distance of the reference shape 20 is maintained in the target shape 201, so that wall thickness along the direction indicated by reference numeral 27 of the reference shape is mapped into the direction of the depth dimension of the cylinder, as indicated by reference numeral 28.

As a result, the left ventricle may be mapped onto the target shape 201 in the form of a cylinder and where the dimension along the cylinder axis represents the thickness of the myocardial wall. The inner reference surface 22 (e.g., the inner wall or endocardium) thus may be projected onto the bottom of the cylinder, i.e. the first surface 29, whereas the and the outer reference surface 23 (e.g., the outer wall or epicardium) may be projected onto the top of the cylinder, i.e., the second surface 200. The tissue between the endocardium and the epicardium is projected onto the planes extending from the bottom to the top of the cylinder.

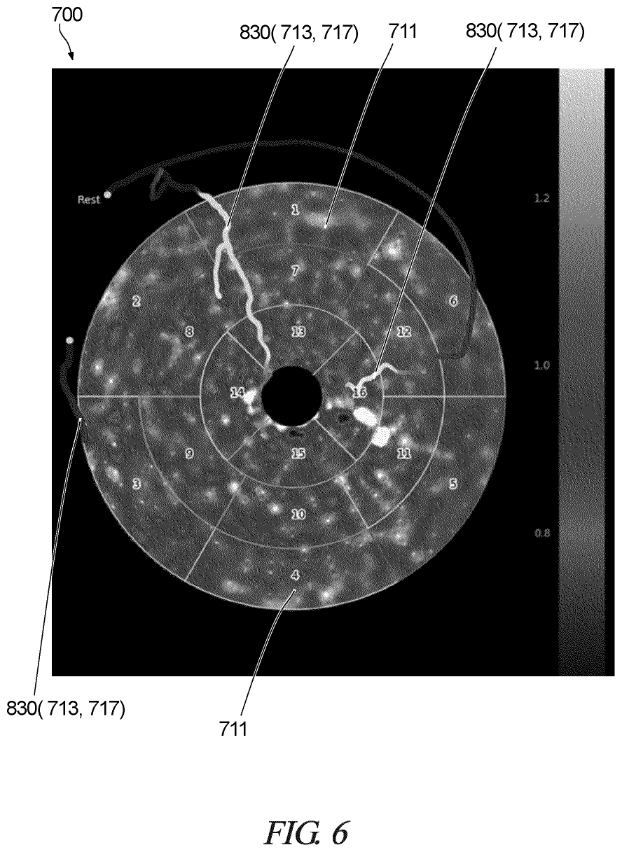

FIG. 6 illustrates visualized perfusion measure and coronary arteries using a bull's eye plot according to one embodiment.

With reference to FIGS. 2A and 6, the block 155 may visualize, on a single image 700, the first perfusion measure 711 of the myocardial tissues 823 and the coronary arteries 830. On the single image 700, the coronary arteries 830 may be overlaid with the first perfusion measure 711. As shown in FIG. 6, the visualized coronary arteries 830 may represent the geometry 713 of the coronary arteries 830 and the flow impediment measure 717 along the coronary arteries 830. In some embodiments, the first perfusion measure 711 and the coronary arteries 830 on the single image 700 may be visualized on a computer screen of the user, or may be stored in a storage. In some embodiments, the first perfusion measure 711 of the myocardial tissues 823 and the coronary arteries 830 that represent the geometry 713 of the coronary arteries 830 and the flow impediment measure 717 along the coronary arteries 830 may be simultaneously visualized on the single image 700, for example on the computer screen of the user. The first perfusion measure 711 and the flow impediment measure 717 may be visualized, for example, at least one of by color, thickness, and size. In the example illustrated in FIG. 6, the first perfusion measure 711 is shown over the annular area including the sections 1-16, and the coronary arteries 830 are shown in the sections 1, 3, 6, 7, 8, 12, 13, 14, and 16 and outside the annular area.

In particular, the complementary imaging features (i.e., the perfusion measure 711 and the coronary arteries 830) may be visualized in the same coordinate system where the intensity in the plane may be an aggregated value of the first perfusion measure 711 throughout the myocardial wall 820. The aggregation can be done by averaging, for example. The width of the coronary arteries 830 may correspond to the effective local cross-sectional area such as to allow for visual stenosis assessment. In some examples, the color of the coronary arteries 830 may encode the flow impediment measure 717.

In some embodiments, the visualization can naturally be enriched by using functional information measured e.g. by invasive FFR, optical coherence tomography (OCT), intravascular ultrasound (IVUS), or X-ray angiography. In some embodiments, the visualization can be augmented by a pixel-to-outlet probability map, where the estimated connection between a vessel and the perfused territory can be seen. In other embodiments, in the single image, stenoses can be particularly highlighted to focus the attention.

In the present disclosure, because the single image 700 may show the first perfusion measure 711 of the myocardial tissues 823, the geometry 713 of the coronary arteries 830, and the flow impediment measure 717 of the coronary arteries 830, the relative contribution of each of the complementary aspects may be judged by the user in a unified graphical figure.

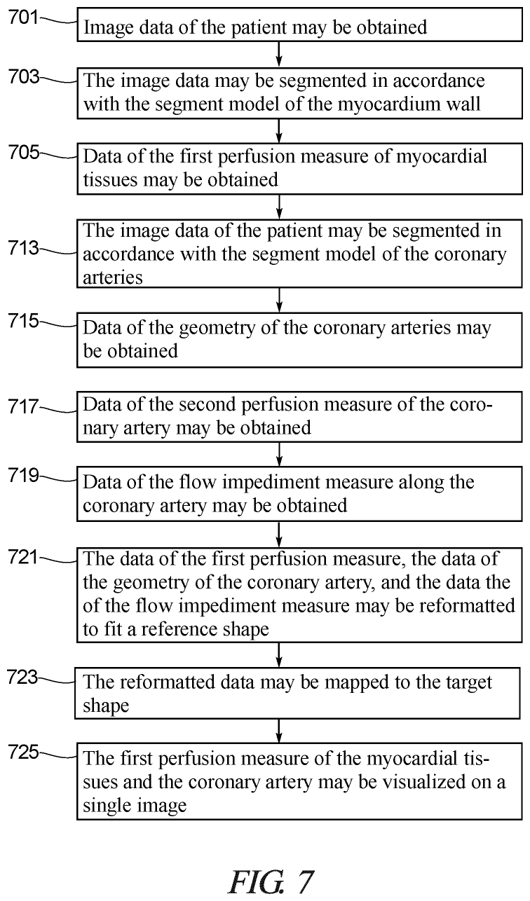

FIG. 7 is a flowchart of a method according to one embodiment of the present disclosure.

In an exemplary method according to one embodiment, in 701 of FIG. 7, the image data 311 of the patient may be obtained. In some embodiments, CCTA data of the patient may be obtained as the image data 311. In 703, the image data 311 of the patient may be segmented in accordance with the segment model of the myocardium wall 820 to provide the segmented data 321 of the myocardium wall 820. In 705, the data 323 of the first perfusion measure 711 of myocardial tissues 823 may be obtained based on the segmented data 321 of the myocardium wall 820.

In 713, the image data 311 of the patient may be segmented in accordance with the segment model of the coronary arteries 830 to provide the segmented data 331 of the coronary arteries 830. In 715, the data 333 of the geometry 713 of the coronary arteries 830 of the patient may be obtained based on the segmented data 331 of the coronary arteries 830. In 717, the data 341 of the second perfusion measure 715 of the coronary arteries 830 may be obtained based on the segmented data 331 of the coronary arteries 830. In 719, the data 343 of the flow impediment measure 717 along the coronary arteries 830 may be obtained based on the data 341 of the second perfusion measure 715 of the coronary arteries 830.

In some embodiments, the processes shown by 713, 715, 717, and 719 may be conducted prior to or in parallel with the processes shown by 703 and 705.

In 721, the data 323 of the first perfusion measure 711 of the myocardial tissues 823, the data 333 of the geometry 713 of the coronary arteries 830, and the data the of the flow impediment measure 717 along the coronary arteries 830 may be reformatted to fit the reference shape 20. In 723, the reformatted data 325 of the myocardial tissues 823, the reformatted data 335 of the geometry 713 of the coronary arteries 830, and the reformatted data 345 of the flow impediment measure 717 along the coronary arteries 830 may be mapped to the target shape 201. Then, in 725, based on the mapping, the first perfusion measure 711 of the myocardial tissues 823 and the coronary arteries 830 may be visualized on the single image 700. On the single image 700, the coronary arteries 830 may be overlaid with the first perfusion measure 71.

The methods according to the present disclosure may be implemented on a computer as a computer implemented method, or in dedicated hardware, or in a combination of both. Executable code for a method according to the present disclosure may be stored on a computer program product. Examples of computer program products include memory devices, optical storage devices, integrated circuits, servers, online software, etc. Preferably, the computer program product may include non-transitory program code stored on a computer readable medium for performing a method according to the present disclosure when said program product is executed on a computer. In an embodiment, the computer program may include computer program code adapted to perform all the steps of a method according to the present disclosure when the computer program is run on a computer. The computer program may be embodied on a computer readable medium.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

1. A computer-implemented method for visualization, comprising:
obtaining data of a first perfusion measure of myocardial tissues of a patient;
obtaining data of a geometry of a coronary artery of the patient;
obtaining data of a second perfusion measure of the coronary artery;
obtaining data of a flow impediment measure along the coronary artery based on the data of the second perfusion measure of the coronary artery; and
visualizing, on a single image, the first perfusion measure of the myocardial tissues and the coronary artery, the coronary artery being overlaid with the first perfusion measure on the single image, the visualized coronary artery representing the geometry of the coronary artery and the flow impediment measure along the coronary artery.

2. The method of claim 1, wherein the obtaining of the data of the flow impediment measure along the coronary artery includes:
determining a flow deviation of the second perfusion measure from a reference value along the coronary artery; and
processing a stenosis assessment along the coronary artery based on the determined flow deviation to obtain the data of the flow impediment measure.

3. The method of claim 2, wherein the processing of the stenosis assessment along the coronary artery includes:
determining a plurality of stenosis rates along the coronary artery based on the flow deviation; and
processing a cumulative sum of the stenosis rates to obtain the data of the flow impediment measure.

4. The method of claim 1, wherein the visualizing includes simultaneously visualizing on the single image the first perfusion measure of the myocardial tissues and the coronary artery that represents the geometry of the coronary artery and that represents the flow impediment measure along the coronary artery.

5. The method of claim 1, wherein the visualizing includes visualizing the first perfusion measure and the flow impediment measure by at least one of color, thickness, and size.

6. The method of claim 1, further comprising obtaining coronary computed tomography angiography data of the patient,
wherein the obtaining of the data of the first perfusion measure, the obtaining of the data of the geometry of the coronary artery, and the obtaining of the data of the flow impediment measure along the coronary artery are based on the same coronary computed tomography angiography data.

7. The method of claim 1, further comprising:
segmenting image data of the patient in accordance with a segment model of a myocardium wall to provide segmented data of the myocardium wall, wherein the obtaining of the data of the first perfusion measure of the myocardial tissues is based on the segmented data of the myocardium wall;
segmenting the image data of the patient in accordance with a segment model of the coronary artery to provide segmented data of the coronary artery, wherein the obtaining of the data of the geometry of the coronary artery of the patient and the obtaining of the data the flow impediment measure along the coronary artery are based on the segmented data of the coronary artery;
reformatting the data of the first perfusion measure of the myocardial tissues, the data of the geometry of the coronary artery, and the data of the flow impediment measure along the coronary artery, to fit a reference shape, wherein the reference shape is defined by at least an inner reference surface and an outer reference surface.

8. The method of claim 7, further comprising:
mapping, to a target shape, the reformatted data of the myocardial tissues, the reformatted data of the geometry of the coronary artery, and the reformatted data of the flow impediment measure along the coronary artery, wherein the target shape is defined by at least a first target surface and a second target surface, wherein the mapping includes mapping from the inner reference surface to the first target surface and from the outer reference surface to the second target surface; and
based on the mapping, visualizing, on the single image, the first perfusion measure of the myocardial tissues and the coronary artery that represents the geometry of the coronary artery and that represents the flow impediment measure along the coronary artery.

9. A visualization system, comprising:
a memory that stores a plurality of instructions; and
processor circuitry that couples to the memory and is configured to execute the instructions to:
obtain data of a first perfusion measure of myocardial tissues of a patient;
obtain data of a geometry of a coronary artery of the patient;
obtain data of a second perfusion measure of the coronary artery;
obtain data of a flow impediment measure along the coronary artery based on the data of the second perfusion measure of the coronary artery; and
visualize, on a single image, the first perfusion measure of the myocardial tissues and the coronary artery, the coronary artery being overlaid with the first perfusion measure on the single image, the visualized coronary artery representing the geometry of the coronary artery and the flow impediment measure along the coronary artery.

10. The visualization system of claim 9, wherein the processor circuitry is further configured to:
determine a flow deviation of the second perfusion measure from a reference value along the coronary artery; and
process a stenosis assessment along the coronary artery based on the determined flow deviation to obtain the data of the flow impediment measure.

11. The visualization system of claim 10, wherein the processor circuitry is further configured to:
determine a plurality of stenosis rates along the coronary artery based on the flow deviation; and
process a cumulative sum of the stenosis rates to obtain the data of the flow impediment measure.

12. The visualization system of claim 9, wherein the visualizing includes simultaneously visualizing on the single image the first perfusion measure of the myocardial tissues and the coronary artery that represents the geometry of the coronary artery and that represents the flow impediment measure along the coronary artery.

13. The visualization system of claim 9, wherein the visualizing includes visualizing the first perfusion measure and the flow impediment measure by at least one of color, thickness, and size.

14. The visualization system of claim 9, further comprising an imaging device that is configured to obtain coronary computed tomography angiography data of the patient,
wherein the processor circuitry is configured to obtain the data of the first perfusion measure, the data of the geometry of the coronary artery, and the data of the flow impediment measure along the coronary artery, based on the same coronary computed tomography angiography data.

15. A non-transitory computer-readable medium having one or more executable instructions stored thereon, which, when executed by processor circuitry, cause the processor circuitry to perform a method for visualization, comprising:
obtaining data of a first perfusion measure of myocardial tissues of a patient;
obtaining data of a geometry of a coronary artery of the patient;
obtaining data of a second perfusion measure of the coronary artery;
obtaining data of a flow impediment measure along the coronary artery based on the data of the second perfusion measure of the coronary artery; and
visualizing, on a single image, the first perfusion measure of the myocardial tissues and the coronary artery, the coronary artery being overlaid with the first perfusion measure on the single image, the visualized coronary artery representing the geometry of the coronary artery and the flow impediment measure along the coronary artery.
